(19) United States
(12) Patent Application Publication
(10) Pub. No.: US 2007/0243624 A1
(43) Pub. Date: Oct. 18, 2007

(54) HYDROGEN EMISSION REPORTING SYSTEM AND METHOD THEREOF

(75) Inventors: A. John Speranza, West Hartford, CT (US); William Raymond Bailey III, West Hartford, CT (US); Angelo Alberto Morson, South Windsor, CT (US); Lawrence Clinton Moulthrop, Windsor, CT (US)

Correspondence Address: PROTON ENERGY SYSTEM, 10 TECHNOLOGY DRIVE, WALLINGFORD, CT, 06492, US

(73) Assignee: Proton Energy Systems Inc., Wallingford, CT (US)

(21) Appl. No.: 11/279,418

(22) Filed: Apr. 12, 2006

(51) Int. Cl. G01N 33/00

(52) U.S. Cl. 436/144

(57) ABSTRACT

The present invention provides a system and method for monitoring and reporting the controlled and uncontrolled emissions of hydrogen gas from an end use application such as a hydrogen cooled electrical generator. The system first determines a quantity hydrogen produced by a hydrogen conversion device from a first operating parameter. The quantity of hydrogen released in a controlled manner is determined from a second operating parameter. The amount of uncontrolled hydrogen emissions may then be calculated from the produced hydrogen and controlled release values. A report detailing the type and amounts of hydrogen emissions may then be created.

### FIELD OF INVENTION

This disclosure relates generally to a system for determining controlled and uncontrolled emissions from a process using hydrogen gas, and more particularly to a system for providing real time reporting of controlled and uncontrolled hydrogen emissions to satisfy emission compliance standards.

### BACKGROUND OF THE INVENTION

Modern societies are critically dependent on energy derived from fossil fuels to maintain their standard of living. As more societies modernize and existing modern societies expand, the consumption of fossil fuels continues to increase and the growing dependence worldwide on the use of fossil fuels is leading to a number of problems. First, fossil fuels are a finite resource and concern is growing that fossil fuels will become fully depleted in the foreseeable future. Scarcity raises the possibility that escalating cost could destabilize economies. Second, fossil fuels are highly polluting. The greater combustion of fossil fuels has prompted recognition of global warming and the dangers it poses to the environment. In order to prevent the deleterious effects of pollution caused by fossil fuels and other materials, various regulatory procedures have been put in place to track emissions of chemicals into the atmosphere.

One common process material used in industrial and energy applications such as electrical power generation is hydrogen gas. To improve the efficiency and reliability of electrical power generators, hydrogen gas is used as a working fluid in the casing of the generator windings. By operating the windings in a hydrogen atmosphere, the aerodynamic drag on the windings is reduced, resulting in increased efficiency of the generator. Since hydrogen gas also transfers heat better than air, the generator may operate at lower temperatures and impose a reduced load on the plant cooling systems.

Unfortunately, due to the small size of the hydrogen molecule, containing pressurized hydrogen gas in an electrical generator is a challenge. While improvements have been made to the sealing assemblies in electrical generators, leaks still exist or appear due to normal wear, resulting in a loss of hydrogen gas. Additionally, over time, the hydrogen gas will become contaminated with oils and water. These contaminants reduce the efficiency of the electrical generator.

To resolve the contamination issue, a portion of the hydrogen gas will typically be removed and processed through a purification system such as a swing bed or heated dryer that uses filters and desiccants to remove the impurities. This purification process inevitably results in additional fittings and connections to the generator that are prone to leak the hydrogen gas.

Since the regulatory agencies are expected to require close tracking and reporting of all emissions, including hydrogen emissions, from electrical power generation facilities and other industrial facilities, a system for monitoring these emissions needs to be created. One method currently proposed uses a mass flow meter coupled to the hydrogen inlet to the generator and the hydrogen outlet. Mass flow meters, however, are expensive, difficult to calibrate and are subject to contamination. These attributes, while solvable through preventative maintenance and periodic testing, are less than acceptable to plant operators. While these existing prior art systems have been used to monitor emissions and were acceptable for their intended purpose, a new method and system is needed to assure accurate monitoring and reporting of hydrogen emissions in a cost effective manner with minimal involvement from the process operator.

### SUMMARY OF THE INVENTION

The present invention provides a method for reporting emissions for a process using hydrogen gas. The method determines what quantity of hydrogen is produced by a hydrogen conversion device by measuring a first operating parameter. The quantity of hydrogen purposely released in a controlled manner is determined by measuring a second operating parameter. The amount of uncontrolled hydrogen emissions is calculated from the quantity of hydrogen generated and the quantity of hydrogen purposely released. The value representing the quantity of uncontrolled hydrogen emissions is stored and transmitting the value representative of the quantity of uncontrolled hydrogen emissions.

A system is also provided for generating a report on emissions from a process using hydrogen gas. The system includes at least one computer processor means for processing data and a storage means for storing data on a storage medium. A second means for processing data is provided for determining the amount of hydrogen gas generated from a first hydrogen conversion device operating parameter and accumulating the amount of hydrogen gas produced. A third means for processing data is also provided for determining the amount of hydrogen gas released using a second operating parameter and accumulating the amount of hydrogen gas purposely released. Lastly, a fourth means for processing data is provided for determining the aggregate controlled and uncontrolled hydrogen emissions that result from the process using hydrogen gas.

A system for providing a report on emissions from a hydrogen cooled electrical generator is also provided. The system includes a hydrogen conversion device with a sensor. A hydrogen cooled electrical generator coupled to the hydrogen conversion device. A vent is coupled to the electrical generator where the vent has means for limiting hydrogen gas flow. Finally, a first controller is electrically coupled to the means for limiting vent hydrogen gas flow. The first controller includes means for determining the flow of hydrogen based on the operating state of the means for limiting vent hydrogen gas flow.

The above discussed and other features will be appreciated and understood by those skilled in the art from the following detailed description and drawings.

### BRIEF DESCRIPTION OF THE DRAWINGS

Referring now to the drawings, which are meant to be exemplary and not limiting, and wherein like elements are numbered alike:

FIG. 1 is a schematic illustration of the system for providing the monitoring and reporting hydrogen gas emissions in an end use application in accordance with an embodiment of the invention;

FIG. 2 illustrates a schematic diagram of a partial electrochemical cell showing an electrochemical reaction for use in accordance with an embodiment of the invention;

FIG. 3 is a state transition diagram illustrating an exemplary embodiment for control methodology in monitoring and reporting hydrogen gas emissions shown in FIG. 1;

FIG. 4 illustrates a schematic diagram of a controller use in the system for providing monitoring and reporting hydrogen emissions shown in FIG. 1;

FIG. 5 illustrates a schematic diagram of an alternate embodiment ventilation arrangement including a sensor for measuring a characteristic of the vent hydrogen gas stream.

### DESCRIPTION OF PREFERRED EMBODIMENT

Disclosed herein are embodiments for a system that provides monitoring and reporting of hydrogen gas emissions from a process that produces and utilizes hydrogen gas such as is used with hydrogen cooled electrical generators.

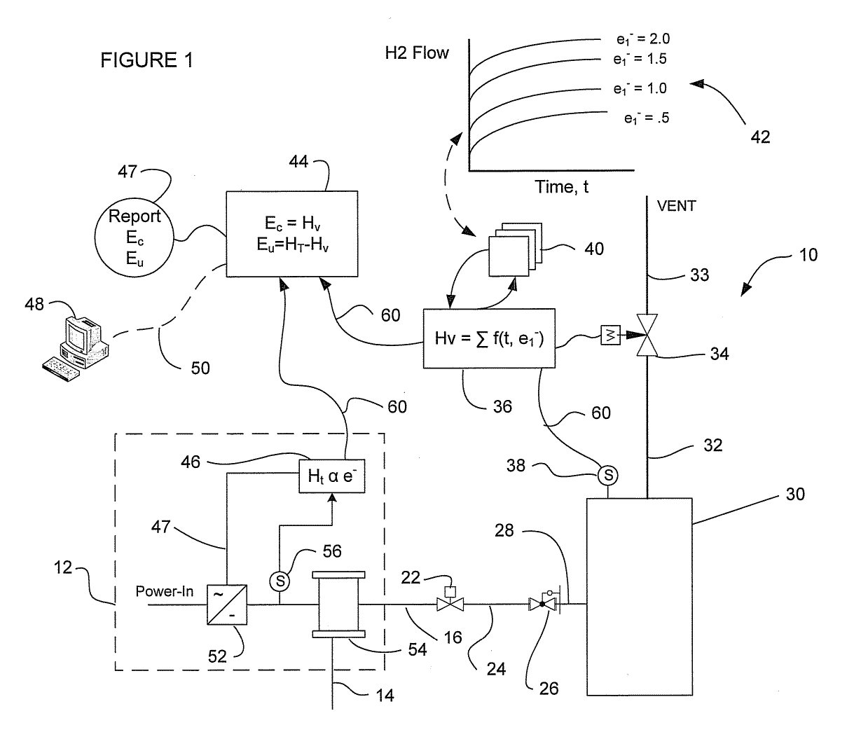

Referring to FIG. 1, a system 10 that uses hydrogen gas is shown. The system 10 includes a hydrogen conversion device 12 that receives a precursor material 14 that is converted to provide hydrogen gas. The hydrogen gas exits the hydrogen conversion device 12 through a conduit 16 to valve 22.

During normal operation, the hydrogen gas will proceed through valve 22 to conduit 24 and through optional pressure reducing regulator 26. Pressure reducing regulator 26 lowers the pressure of the hydrogen gas from its production pressure to the application pressure needed by the end use application. After the pressure of the gas is reduced, the hydrogen gas stream enters conduit 28. Conduit 28 terminates at the end use application 30, such as a hydrogen cooled electrical power generator. Optionally, a compressor (not shown) may be utilized to increase the pressure of the hydrogen gas where the hydrogen conversion device 12 produces the gas at a pressure that is less than the needs of end use application 30. It should also be appreciated that additional valves, conduits, tanks and fluid control equipment may be incorporated into the system 10 as is needed to provide a supply of hydrogen to the end use application 30 without departing from the spirit or intent of the present invention.

The hydrogen gas flows from conduit 28 into the end use application 30. In the preferred embodiment, the end use application 30 is an electrical power generator 30 that utilizes the hydrogen gas as an atmosphere in which the generator windings operate. The use of hydrogen gas provides a number of advantages in improving efficiency and heat transfer. However, the end use application may be any process that utilizes hydrogen gas as a processing or working fluid during operation, for example, the end use application may be a hydrogen furnace that is used in the treating of metals or the processing of silicon semiconductors. Another example of such an application would be a hydrogen torch used in certain brazing applications in the manufacture of optical components. These alternate end use applications are provided as exemplary embodiments only and are not intended to limit the scope of the present invention.

The end use application 30 includes a vent that couples with conduit 32 to allow a controlled release of hydrogen gas from the end use application 30. The flow of hydrogen gas through conduit 32 is regulated by valve 34. In the preferred embodiment, valve 34 is a variable opening valve that is controlled by an actuator such as a motor or a solenoid. The valve 34 may be a needle valve capable of opening in discrete increments from fully closed to fully open. The valve 34 actuator is operated by a signal from a controller 36. As will be described in more detail below, controller 36 actuates the valve 34 in response to a signal from sensor 38 that is coupled to the end use application 30. The sensor 38 is capable of sensing appropriate parameters that are important to the end use application. In the preferred embodiment hydrogen cooled electrical power generator, the sensor 38 monitors hydrogen purity, meaning percentage of hydrogen gas contained in the electrical generator 30. Other examples of sensors 38 may include dew point sensors or pressure transducers. It should be appreciated that while the system 10 is described in reference to a single one sensor, any number of sensors measuring different criteria that affect the end use application 30 may be utilized either singly as described or in combination as needed to improve the operation of end use application 30.

The controller 36 receives the signal from sensor 38 and adjusts the hydrogen flow rate in conduit 32 to achieve the desired hydrogen gas operating state in the end use application 30. Typically, the controller changes the position of the valve 34 by changing the voltage e₁⁻ and/or current applied to the valve 34 actuator. In the exemplary embodiment shown in FIG. 1, the valve 34 includes five operating positions, e.g. e₁⁻=0V, e₁⁻=0.5V, e₁⁻=1.0V, e₁⁻=1.5V, and e₁⁻=2.0V. Any number operating positions may be utilized by the present invention, and the number may be increased or decreased depending on the needs of the operator of end use application 30. In the preferred embodiment, the controller 36 includes look-up tables 40 that allow the controller to calculate the amount of controlled hydrogen emissions released through conduit 33. The look-up tables 40 are numerical representation of flow curves 42 for each valve position.

Alternatively, the valve 34 may be replaced by other fluid control devices that allow the controller to calculate the volume of hydrogen that flows through conduits 32, 33. For example, the valve 34 may be replaced by a manifold containing a plurality of fixed orifices and appropriate valves to route the hydrogen gas. In this alternate embodiment, the controller 36 changes the valve settings in the manifold to achieve the desired hydrogen flow. Alternately, the valve 34 may be replaced by a solenoid valve pulsed on and off at a controlled rate to effect a controlled flow rate or through the use of similar valves such as a PWM controlled valve.

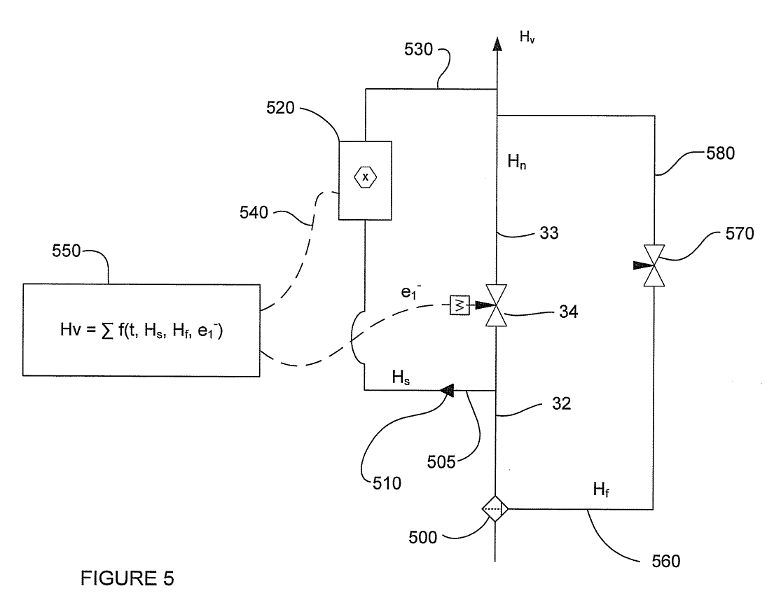

Another alternate embodiment is shown in FIG. 5. In this embodiment, either an additional sensor or an alternative sensor 520 is coupled to the hydrogen vent conduit 32. In this embodiment, a sample of the hydrogen gas is drawn from conduit 32 into conduit 505. A flow limiting device 510, such as a fixed orifice, or a needle valve, restricts the flow of hydrogen Hₛ through conduit 505 to sensor 520. In the exemplary embodiment, the sensor 520 measures the hydrogen purity of the hydrogen gas in the vent conduit 32. Sensor 520 provides a signal via line 540 to controller 550. Controller 550 then adjusts the valve 34 as described above. Optionally, one or more coalescing filters 500 may be coupled to the conduit 32 to remove water from the hydrogen gas in prior to sampling the hydrogen gas stream. Where the optional coalescing filter 500 is included, the hydrogen gas pressure is utilized to force the water trapped in coalescing filter 500 through conduit 560. The flow of water and hydrogen gas H_{f} through conduit 560 is restricted by valve 570. The hydrogen gas H_{f} passes through the valve 560 into conduit 580 where it is returned to the vent conduit 33. It should be appreciated that the hydrogen gas flows Hₛ and H_{f} are generally fixed or constant values. These values Hₛ and H_{f} represent additional controlled hydrogen gas emissions that need to be accounted for in controller 550. In the exemplary embodiment, the sensor hydrogen gas flow Hₛ is between 0.25 liters/minute and 1 liter/minute while the filter hydrogen gas flow H_{f} is approximately 0.5 liters/minute.

The controller 36 accumulates the controlled hydrogen emission data and passes the data to a controller 44. As will be described in more detail below, the controller 44 combines the data from controller 36 with hydrogen generation data from controller 46 in the hydrogen conversion device 12. From the received data, the controller 44 determines the total controlled hydrogen emissions E_{c} and uncontrolled hydrogen emissions Eᵤ. Controller 44 may issue a report 47 or further communicate with other remote computers 48 on a network 50. In addition to being coupled to one or more components within system 10 and remote computers 48, controller 44 may also be coupled to external computer networks such as a local area network (LAN) and the Internet. LAN 48 interconnects one or more remote computers, which are configured to communicate with controller 44 using a well known computer communications protocol such as TCP/IP (Transmission Control Protocol/Internet Protocol), RS-232, ModBus, and the like. Alternatively, a proprietary communications protocol may be used.

Alternately, the controllers 46 and 36 may transmit flow data 60 via analog signals representative of the flow rate of hydrogen produced and the flow rate of hydrogen purposely vented. Process monitoring computer 44 may then process this data to determine E_{c} and Eᵤ and report same.

Typically, prior art systems 10 utilized compressed hydrogen gas stored in cylinders that were periodically replaced as the supply of hydrogen was expended. Since these cylinders were heavy, bulky and required special care to avoid safety issues, operators of end use applications have found it advantageous to utilize technologies that generate hydrogen gas as needed rather than relying on the storage means. These on-site hydrogen generation devices, such as hydrogen conversion device 12, consume a precursor material that is converted into hydrogen gas and typically a byproduct material. The precursor material may take many forms, such as water, algae, bacteria, hydrocarbons, oxygenated hydrocarbons, organic carbon materials, fixed metal hydrides, transferable metal hydrides, and sodium borohydroxide. Typically, a different process is used for each precursor material, as will be described in more detail below, the hydrogen conversion device may include alkaline electrochemical cells, phosphoric acid electrochemical cells, solid oxide electrochemical cells, proton exchange membrane electrochemical cells, steam methane reformer, natural gas reformer, coal reformer, hydrocarbon reformer, partial oxidation reactors, ceramic membrane reactor, photolysis reactor, photoelectrolysis reactor, photochemical reactors, photobiological reactors, anaerobic digesters or bio-mass gasification reactors.

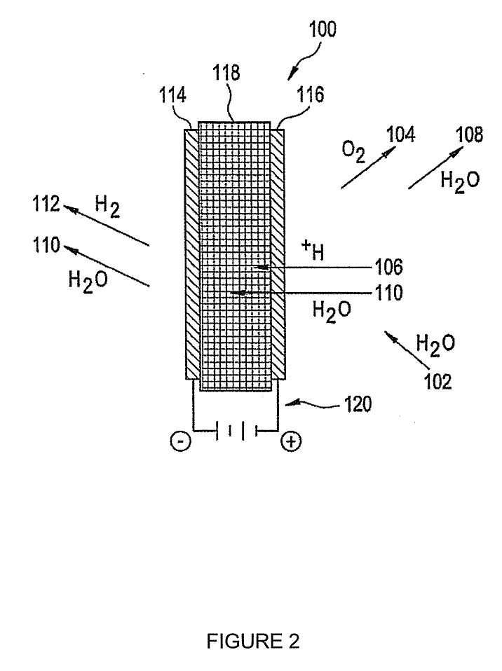

Electrochemical cells are energy conversion devices that may be classified as electrolysis cells or fuel cells. A proton exchange membrane electrolysis cell can function as a hydrogen generator by electrolytically decomposing water to produce hydrogen and oxygen gas, and can function as a fuel cell by electrochemically reacting hydrogen with oxygen to generate electricity. Referring to FIG. 2, which is a partial section of a typical anode feed electrolysis cell 100, process water 102 is fed into cell 100 on the side of an oxygen electrode (anode) 116 to form oxygen gas 104, electrons, and hydrogen ions (protons) 106. The reaction is facilitated by the positive terminal of a power source 120 electrically connected to anode 116 and the negative terminal of power source 120 connected to a hydrogen electrode (cathode) 114. The oxygen gas 104 and a portion of the process water 108 exits cell 100, while protons 106 and water 110 migrate across a proton exchange membrane 118 to cathode 114 where hydrogen gas 112 is formed.

Another typical water electrolysis cell using the same configuration as is shown in FIG. 2 is a cathode feed cell, wherein process water is fed on the side of the hydrogen electrode. A portion of the water migrates from the cathode across the membrane to the anode where hydrogen ions and oxygen gas are formed due to the reaction facilitated by connection with a power source across the anode and cathode. A portion of the process water exits the cell at the cathode side without passing through the membrane.

Electrochemical cell systems typically include a number of individual cells arranged in a stack, with the working fluids directed through the cells via input and output conduits formed within the stack structure. The cells within the stack are sequentially arranged, each including a cathode, a proton exchange membrane (PEM), and an anode. The cathode and anode may be separate layers or may be integrally arranged with the membrane. Each cathode/membrane/anode assembly (hereinafter “membrane electrode assembly”, or “MEA”) typically has a first flow field in fluid communication with the cathode and a second flow field in fluid communication with the anode. The MEA may furthermore be supported on both sides by screen packs or bipolar plates disposed within flow fields. Screen packs or bipolar plates may facilitate fluid movement to and from the MEA, membrane hydration, and may also provide mechanical support for the MEA.

In the preferred embodiment, hydrogen conversion device 12 includes the membrane 118 comprised of electrolytes that are preferably solids or gels under the operating conditions of the electrochemical cell. Useful materials include proton conducting ionomers and ion exchange resins. Useful proton conducting ionomers include complexes comprising an alkali metal salt, alkali earth metal salt, a protonic acid, or a protonic acid salt. Useful complex-forming reagents include alkali metal salts, alkaline metal earth salts, and protonic acids and protonic acid salts. Counter-ions useful in the above salts include halogen ion, perchloric ion, thiocyanate ion, trifluoromethane sulfonic ion, borofluoric ion, and the like. Representative examples of such salts include, but are not limited to, lithium fluoride, sodium iodide, lithium iodide, lithium perchlorate, sodium thiocyanate, lithium trifluoromethane sulfonate, lithium borofluoride, lithium hexafluorophosphate, phosphoric acid, sulfuric acid, trifluoromethane sulfonic acid, and the like. The alkali metal salt, alkali earth metal salt, protonic acid, or protonic acid salt is complexed with one or more polar polymers such as a polyether, polyester, or polyimide, or with a network or cross-linked polymer containing the above polar polymer as a segment. Useful polyethers include polyoxyalkylenes, such as polyethylene glycol, polyethylene glycol monoether, and polyethylene glycol diether; copolymers of at least one of these polyethers, such as poly(oxyethylene-co-oxypropylene)glycol, poly(oxyethylene-co-oxypropylene)glycol monoether, and poly(oxyethylene-co-oxypropylene)glycol diether; condensation products of ethylenediamine with the above polyoxyalkylenes; and esters, such as phosphoric acid esters, aliphatic carboxylic acid esters or aromatic carboxylic acid esters of the above polyoxyalkylenes. Copolymers of, e.g., polyethylene glycol with dialkylsiloxanes, maleic anhydride, or polyethylene glycol monoethyl ether with methacrylic acid are known in the art to exhibit sufficient ionic conductivity to be useful.

Ion-exchange resins useful as proton conducting materials include hydrocarbon- and fluorocarbon-type resins. Hydrocarbon-type ion-exchange resins include phenolic resins, condensation resins such as phenol-formaldehyde, polystyrene, styrene-divinyl benzene copolymers, styrene-butadiene copolymers, styrene-divinylbenzene-vinylchloride terpolymers, and the like, that are imbued with cation-exchange ability by sulfonation, or are imbued with anion-exchange ability by chloromethylation followed by conversion to the corresponding quaternary amine.

Fluorocarbon-type ion-exchange resins can include hydrates of tetrafluoroethylene-perfluorosulfonyl ethoxyvinyl ether or tetrafluoroethylene-hydroxylated (perfluoro vinyl ether)copolymers. When oxidation and/or acid resistance is desirable, for instance, at the cathode of a fuel cell, fluorocarbon-type resins having sulfonic, carboxylic and/or phosphoric acid functionality are preferred. Fluorocarbon-type resins typically exhibit excellent resistance to oxidation by halogen, strong acids and bases. One family of fluorocarbon-type resins having sulfonic acid group functionality is NAFION™ resins (commercially available from E. I. du Pont de Nemours and Company, Wilmington, Del.).

Electrodes 114 and 116 comprise a catalyst suitable for performing the needed electrochemical reaction (i.e., electrolyzing water and producing hydrogen). Suitable catalyst include, but are not limited to, materials comprising platinum, palladium, rhodium, carbon, gold, tantalum, tungsten, ruthenium, iridium, osmium, alloys of at least one of the foregoing catalysts, and the like. Electrodes 114 and 116 can be formed on membrane 118, or may be layered adjacent to, but in contact with, membrane 118.

Alternatively, the hydrogen conversion device 12 may be a simplified electrochemical compressor-type cell. In this embodiment, a hydrogen-water precursor material is introduced into an electrochemical cell having electrodes 114 and 116 comprising a platinum catalyst. The hydrogen in the precursor is permeated through the membrane 118 separating the hydrogen from the water and optionally allowing the hydrogen pressure to be increased simultaneously.

Alternatively, the hydrogen conversion device 12 may be a biomass reactor that uses chemical and/or electrochemical reactions in the production of hydrogen gas. These processes include naturally occurring organic matter with a base. Preferably, the organic matter is biomass. Biomass is a general term used to refer to all non-fossil organic materials that have intrinsic chemical energy content. Biomass includes organic plant matter, vegetation, trees, grasses, aquatic plants, wood, fibers, animal wastes, municipal wastes, crops and any matter containing photosynthetically fixed carbon. Biomass is available on a renewable or recurring basis and is thus much more readily replenished than fossil fuels. The volume of biomass available makes it a naturally occurring carbon resource that is sufficiently plentiful to substitute for fossil fuels.

The capture of solar energy through photosynthesis drives the formation of biomass. During photosynthesis, the organic compounds that make up biomass are produced from CO2 and H2O in the presence of light. The principle compounds present in biomass are carbohydrates. Glucose (C2H12O6) is a representative carbohydrate found in biomass and is formed in photosynthesis through the reaction:
6CO₂+6H₂O [symbol] C₆H₁₂O₆+6O₂

Reactions of organic substances with a base permit the production of hydrogen gas through the formation of carbonate ion and/or bicarbonate by-products. Inclusion of a base as a reactant in the production of hydrogen from organic substances thus provides a reaction pathway for the production of hydrogen. For example, hydrogen may be produced from glucose (C₆H₁₂O₆) through exposure to high temperatures in the following reaction:
C₆H₁₂O₆+6H₂O⇄6CO₂+12H₂

This reaction is representative of reformation reactions of organic substances that are analogous to those used in the reformation of simple compounds such as methanol or ethanol. In another example, hydrogen may be produced from sucrose by reacting it with a base such as sodium hydroxide (NaOH). Representative reactions of sucrose with sodium hydroxide are given below:
C₁₂H₂₂O₁₁+24NaOH+H₂O⇄12Na₂CO₃+24H₂
or
C₁₂H₂₂O₁₁+12NaOH+13H₂O⇄12NaHCO₃+24H₂

Alternatively, similar to the biomass reformation, the hydrogen conversion device 12 may create hydrogen gas through a reaction of carbonaceous matter, such as coal, with a base. Carbonaceous matter refers generally to naturally occurring carbon-containing materials and substances such as coal. Coal typically includes carbon along with various organic and inorganic compounds or elements. Various coals may be used as starting materials in hydrogen-producing reactions, including anthracitic, bituminous, sub-bituminous, and lignitic coals. The primary constinutents of coal are carbon, hydrogen, nitrogen, oxygen and sulfur.

Base facilitated reactions lead to the production of hydrogen from carbonaceous matter. The reactions provide an alternate pathway of carbonaceous matter that leads to a more spontaneous reaction at a particular set of reaction conditions to permit the liberation of hydrogen contained in the carbonaceous matter as hydrogen gas. For example:
C₍ₛ₎+H₂O⇄CO+H₂

Where C₍ₛ₎ refers to the carbon contained in coal. The product mixture of carbon monoxide and H₂ gases are known as syngas and can be further reacted to produce other hydrogenated organic fuels such as methanol or ethanol. The carbon monoxide of syngas can be reacted via the water-gas shift reaction to produce additional hydrogen:
CO+H₂O⇄CO₂+H₂

By combining these reactions, a net coal reaction may be written as:
C₍ₛ₎+2H₂O⇄CO₂+2H₂

As with biomass, the addition of a base may also facilitate the generation of hydrogen gas through a reaction that also produces a carbonate or bicarbonate by-product. When sodium hydroxide is combined with the coal, for example:
C₍ₛ₎+2NaOH+H₂O⇄2H₂+NaCO₃
or
C₍ₛ₎+NaOH+2H₂O⇄2H₂+NaHCO

Here, the reaction of a base with coal results in the by-products of either sodium carbonate or sodium bicarbonate with the formation of hydrogen gas.

Alternatively, the hydrogen conversion device 12 may produce hydrogen gas through the reformation of an ammonia precursor. Ammonia may be thermo-catalytically cracked at relatively low temperatures to produce a gas mixture that is 75% hydrogen by volume. The ammonia decomposition reaction may be represented as follows:
2NH₃→N₂+3H₂

Generally, trace amounts of un-reacted ammonia remain in the product stream requiring further purification prior to use.

Alternatively, the hydrogen conversion device 10 may produce hydrogen gas through the reformation of a hydro-carbon based precursor such as natural gas, LPG, gasoline, methanol or diesel. This reformation process typically utilizes a multistep process combined with several clean-up processes. The initial step in the process may be steam reformation, catalytic reformation, autothermal reformation or catalytic partial oxidation or non-catalytic partial oxidation. Since trace amounts of the precursor material may be found in the resulting gas stream, clean-up processes including desulfurization, high temperature water-gas shift, low temperature water-gas shift, selective CO oxidation, or selective CO methanation are used. Alternatively, processes such as hydrogen selective membrane reactors and filters may be used.

Steam reformation or steam methane reforming is a method of producing hydrogen through the chemical decomposition of water. The hydrogen gas is produced through the introduction of steam at high temperatures, typically 700-1100° C., to methane in the presence of a metal-based catalyst. Typically, the steam reformation process uses natural gas as the source of the methane. This reaction results in carbon monoxide and hydrogen:
CH₄+H₂O→CO+3H₂

In catalytic reformation, the hydrogen gas is generated as a by-product to the production of gasoline. This process typically uses a bifunctional catalyst is utilized to rearrange and break the hydrocarbon chains from the hydrocarbon fuel (e.g. oil).

In autothermal reformation, a part of the hydrocarbon fuel is oxidized by controlled addition of oxygen in the presence of oxidation catalysts. The energy which is released during oxidation is necessary for the endothermic steam reformation taking place simultaneously. The temperature which results is between that of partial oxidation and that of steam reformation.

Also, hydrogen may be produced from hydrocarbon based precursors utilizing a process called catalytic partial oxidation. In this process, the hydrocarbon material, such as natural gas, is combined in a reactor with oxygen over a solid catalyst bed. When processed at the proper temperature and ratio of reactants the resultant reaction forms carbon monoxide and hydrogen:
CH₄+½O₂→CO+2H₂

Typically, contaminants such as sulfur are also found in the resulting gas stream and require further post processing to remove the contaminants prior to use.

Alternatively, the hydrogen conversion device may disassociate hydrogen from water through a process known as photolysis. In general, photolysis refers to any chemical reaction through which water is disassociated by light. Typically, the disassociation of water is achieved by subjecting the water to ultraviolet (UV) light in the presence of a catalyst such as titanium dioxide, or tantalum oxide with a co-catalyst nickel oxide. The resultant reaction creates both hydrogen and oxygen gas.

Photolysis may also be utilized with algae through the process of photosynthesis to produce hydrogen gas. Typically, the plant utilizes light from the sun and carbon-dioxide from the air to grow new cells. Through modifications in the algae's environment, the algae may be induced to produce hydrogen rather than oxygen. This process, which typically occurs in a bioreactor, the algae are grown and deprived carbon dioxide and oxygen. This deprivation causes a stress on the algae resulting in a dormant gene becoming activated that results in the synthesis of an enzyme called hydrogenase. The algae use this enzyme to produce both hydrogen and oxygen gas from the surrounding water. The process may be enhanced by creating a sulfur deficient environment as well. As with other hydrogen production methods, post generation processing is required to separate the hydrogen from other gases and contaminants.

One issue with all methods of hydrogen gas generation is the presence of undesired compounds in the resulting gas stream. These compounds may be un-reformed precursor material such as methane or water. Other undesired compounds include reformation byproducts such as carbon monoxide or contaminants found in the precursor material (e.g. sulfur). Since end use applications of hydrogen gas may be sensitive to the introduction of these compounds, it is advantageous to provide a means that automatically detects the undesired compound and takes corrective action to prevent issues or damage to the end use application.

One feature that is common to each of the different hydrogen conversion device technologies is that there is a relationship that may be utilized to determine the total amount of hydrogen gas generated by the hydrogen conversion device 12. In the preferred embodiment, the hydrogen conversion device is an electrochemical cell that converts water in to hydrogen and oxygen in the presence of a catalyst and electrical current. The amount of hydrogen produced by an electrochemical cell is determined by the relationship:
H_{generated}=(7.52)(N)(I)−L, cc/min
where N represents the number of cells in the electrochemical cell stack, I represents current bussed through the cells in series, and L represents hydrogen conversion losses (e.g. drying purge losses, hydrogen permeation losses, etc.) that are known by the system architecture and characteristics. Alternatively, the amount of hydrogen produced byan electrochemical cell may be determined on a mass rate basis by the relationship:
Hmass=(0.037384)(N)(I)−L, g/hr
Similar empirical or analytic relationships for other hydrogen conversion devices are also known in the art.

Referring back to FIG. 1, the hydrogen conversion device 12 in the preferred embodiment is an electrochemical cell stack 54, such as an electrolysis cell stack. The hydrogen conversion device 12 receives electrical power from a source, including but not limited to the electrical grid or a renewable power source (e.g. photovoltaic arrays or wind turbines), and converts the electrical power to direct current in converter 52. The electrical current is applied to the electrochemical cell stack 54 where along with water from conduit 14, the hydrogen gas is generated and exits the hydrogen conversion device 12 at a predetermined pressure. The generated pressure may range from 14.7 psia-10,000 psig. In the preferred embodiment, the generated pressure is between 100 psi-225 psi and more preferably 200 psi.

Generally, an hydrogen conversion device 12 will have a feedback control system (not shown) to allow the hydrogen conversion device to adapt its operation to the requirements of the end use application 30 such as that described in U.S. Pat. No. 6,822,205 entitled “Electrochemical cell system output control method and apparatus” which is incorporated herein by reference. In the preferred embodiment, the hydrogen conversion devise 12 will detect a drop in pressure in the end use application 30 and increase the output of hydrogen gas to bring the pressure in the end use application 30 back to the desired operating parameter. The hydrogen conversion device accomplishes this through a control signal 47 to power converter 52. Specifically, the controller 46 determines whether to increase or decrease the current provided to cell stack 54 is required by power converter 52. If the controller 46 determines that there is insufficient hydrogen pressure, power converter 52 is instructed to provide an increase in electrical current to the cell stack 54. An increase in the current to the cell stack 54 will cause an increase in the reaction rate, thereby causing an increase in the pressure in the hydrogen output line 16. If the controller 46 determines there is excessive hydrogen pressure, the power converter is instructed to provide a decrease in electrical current signal to the cell stack 54. In response to receiving the decrease signal 47, power converter 52 decreases current to the cell stack 54. A decrease in the current to cell stack 54 will cause a decrease in the reaction rate, thereby causing a decrease in the pressure in the hydrogen line. It should be recognized that controller 46 performs this process repetitively during the operation of hydrogen conversion device 12.

A sensor 56 monitors the amount of current provided from the converter 52 to the electrochemical cell stack 54 and transmits a signal indicative of the current level to controller 46. Since the amount of hydrogen produced is proportional to the level current provided to the electrochemical cell stack 54, the controller 46 is able to accumulate a total amount of hydrogen produced H_{T}. In the preferred embodiment, the controller 46 periodically or continuously transmits the data H_{T} to the controller 44 to allow calculation of the controlled E_{c} and uncontrolled Eᵤ emissions from the system 10.

In the exemplary embodiment shown in FIG. 1, the controllers 46, 36, 44 are illustrated as three distinct, separate objects. However, it is contemplated that the control functionality described herein may be accomplished by a single or multiple controllers or microprocessors without departing from the present intention. The use of multiple hardware controllers may in some instances be preferable since it would allow the use of self-checking or so called “watch-dog” algorithms to detect hardware malfunctions within the system 10. The use of multiple hardware controllers may also be advantageous where the hydrogen conversion device 12 and the end use application 30 are physically separated from each other. In an embodiment where either a single controller or multiple controllers is used, the controllers 36, 44, 46 are a suitable electronic device capable of accepting data and instructions, executing the instructions to process the data, and presenting, logging, or transmitting the results. Controllers 36, 44, 46 may accept instructions or display information through user interface, or through other means such as but not limited to electronic data card, voice activation means, manually-operable selection and control means, radiated wavelength and electronic or electrical transfer. Therefore, controllers 36, 44, 46 can be a microprocessor, microcomputer, a minicomputer, an optical computer, a board computer, a complex instruction set computer, an ASIC (application specific integrated circuit), a reduced instruction set computer, an analog computer, a digital computer, a molecular computer, a quantum computer, a cellular computer, a superconducting computer, a supercomputer, a solid-state computer, a single-board computer, a buffered computer, a computer network, a desktop computer, a laptop computer, a scientific computer, a scientific calculator, or a hybrid of any of the foregoing.

Controllers 36, 44, 46 are capable of converting the analog voltage or current level provided by an external device such as sensor 38 into a digital signal indicative of the level of purity or impurity in the hydrogen gas stream flowing within the end use application, or a communication from another controller. Alternatively, the external device (e.g. sensor 38) may be configured to provide a digital signal to controllers 36, 44, 46, or an analog-to-digital (A/D) converter (not shown) maybe coupled between an external device and controllers 36, 44, 46 to convert the analog signal provided by external device into a digital signal for processing by controllers 36, 44, 46. Controllers 36, 44, 46 use the digital signals act as input to various processes for controlling aspects of the system 10 operation as well as monitoring and reporting the controlled E_{c} and uncontrolled Eᵤ emissions. The digital signals may represent one or more system 10 data including but not limited to impurity levels, dew point, pressure levels, hydrogen conversion operational state, hydrogen gas flow rate, hydrogen gas pressure, valve 22, 34 operational state and the like.

Controllers 36, 44, 46 are operably coupled with one or more components of system 10 by data transmission media 60. Data transmission media 60 includes, but is not limited to, twisted pair wiring, coaxial cable, and fiber optic cable. Data transmission media 60 also includes, but is not limited to, wireless, radio and infrared signal transmission systems. Controllers 36, 44, 46 are configured to provide operating signals to these components and to receive data from these components via data transmission media 60.

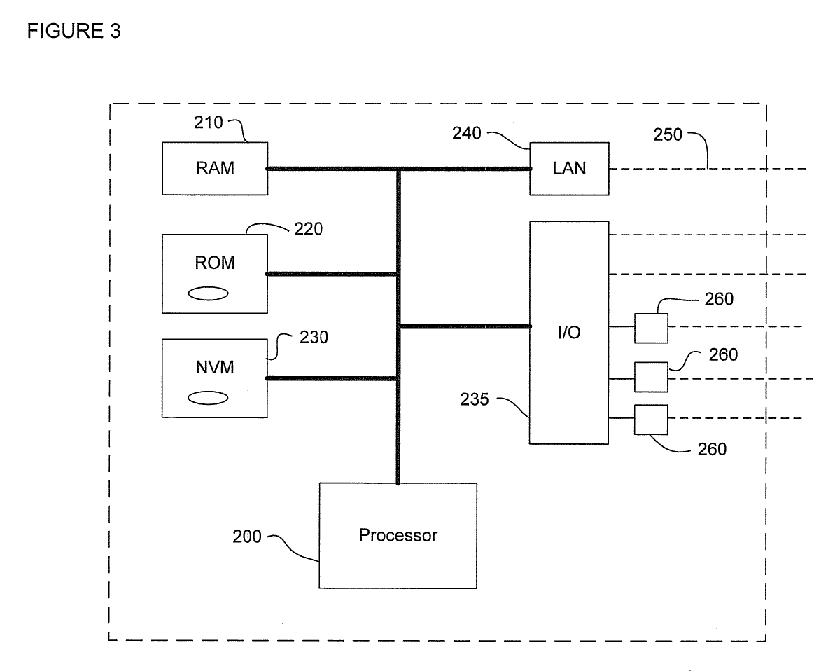

Referring now to FIG. 3, a schematic diagram typical of controllers 36, 44, 46 is shown. Controllers 36, 44, 46 include a processor 200 coupled to a random access memory (RAM) device 210, a non-volatile memory (NVM) device 230, a read-only memory (ROM) device 220, one or more input/output (I/O) controllers 235, and a LAN interface device 240 via a data communications bus 250.

I/O controllers 235 are coupled to external devices including but not limited to sensors 38, 56, and alternatively to a user interface for providing digital data between these devices and bus 250. I/O controllers 235 are also coupled to analog-to-digital (A/D) converters 260, which receive analog data signals from devices including but not limited to sensors 38, 56.

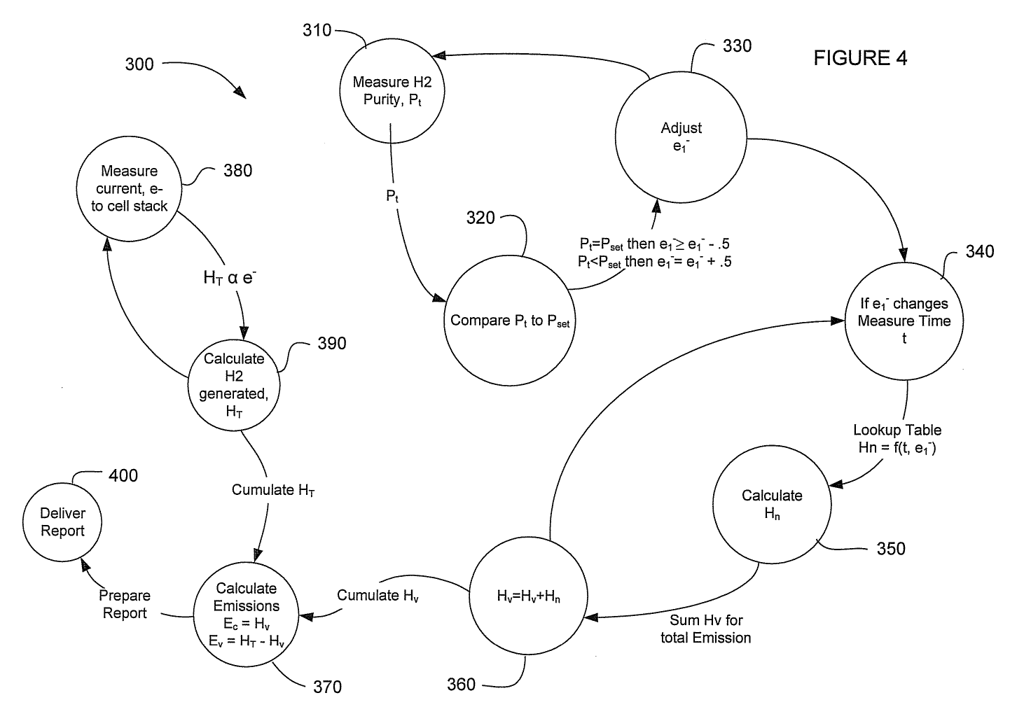

LAN interface device 240 provides for communication between controllers 36, 44, 46 and a local area network (“LAN”) in a data communications protocol supported by the LAN. ROM device 220 stores an application code, e.g., main functionality firmware, including initializing parameters, and boot code, for processor 200. Application code also includes program instructions as shown in FIG. 4 for causing processor 200 to execute any operation control methods, including altering the operation of valve 34, monitoring the amount of hydrogen Hᵥ flowing through the vent conduit 32, monitoring the current applied to the electrochemical cell stack 54, determining the amount controlled E_{c} and uncontrolled Eᵤ hydrogen emissions, and generation of alarms. The data to be exchanged with remote computers 48 and the controller 44 include but are not limited to hydrogen purity levels, dew point, pressure, total emissions, controlled emissions, uncontrolled emissions, operational state of hydrogen conversion device 12 and operational state of valve 34.

NVM device 230 is any form of non-volatile memory such as an EPROM (Erasable Programmable Read Only Memory) chip, a disk drive, or the like. Stored in NVM device 230 are various operational parameters for the application code. The various operational parameters can be input to NVM device 230 either locally, using a keypad or remote computer, or remotely via the Internet using remote computer 48. It should be recognized that application code can be stored in NVM device 230 rather than ROM device 220. Controllers 36, 44, 46 may also be coupled to one or more other storage mediums for the storage of data and application codes. Such alternate storage mediums include, but are not limited to magnetic storage devices (e.g. hard drives, floppy disks), optical storage devices (e.g. compact disk and digital video disk) and solid state storage devices (e.g. flash memory).

Controllers 36, 44, 46 include operation control methods embodied in application code shown in FIG. 4. These methods are embodied in computer instructions written to be executed by processor 200, typically in the form of software. The software can be encoded in any language, including, but not limited to, assembly language, VHDL (Verilog Hardware Description Language), VHSIC HDL (Very High Speed IC Hardware Description Language), Fortran (formula translation), C, C++, Visual C++, Java, ALGOL (algorithmic language), BASIC (beginners all-purpose symbolic instruction code), visual BASIC, ActiveX, HTML (HyperText Markup Language), and any combination or derivative of at least one of the foregoing. Additionally, an operator can use an existing software application such as a spreadsheet or database and correlate various cells with the variables enumerated in the algorithms. Furthermore, the software can be independent of other software or dependent upon other software, such as in the form of integrated software.

Referring to FIG. 4, a state transition diagram depicting an exemplary method of control process 300 for monitoring and reporting the controlled and uncontrolled hydrogen emissions will now be described. The process 300 includes numerous modes and the criterion, requirements, events and the like to control changes of state among the various modes. It should be appreciated that while process 300 will be described sequentially, process 300 includes various processes and modes that exist and operate simultaneously and/or independently of each other.

Measuring mode 310 receives signals from sensor 38 indicative of the level of purity Pₜ in the end use application 30. The process transfers to mode 320 where the detected purity Pₜ is compared with a desired or pre-set purity level Pₛₑₜ. Pₛₑₜ will depend on the needs of the end use application, for example in the exemplary embodiment where the end use application is a hydrogen cooled electrical generator, the operator will typically desire to keep the purity levels in the generator at greater than 98% to maintain the advantages gained through the use of hydrogen. The level set by the operator may also vary depending on other tradeoffs in operation, such as the cost of generating hydrogen. The process 300 will then transition to mode 330 where the operational state of the valve 34 is changed. In the exemplary embodiment, if the detected purity Pₜ is greater than or equal to Pₛₑₜ, then the voltage applied e₁⁻ to the valve actuator is decreased by 0.5V to decrease the amount of hydrogen flowing through conduit 33. If the detected purity Pₜ is less than Pₛₑₜ, then the voltage applied e₁⁻ to the valve actuator is increased by 0.5V is increase the amount of hydrogen flowing in the conduit 33. By increasing the flow of hydrogen through the conduit 33, the pressure level in the end use application 30 will decrease. The drop in pressure is detected by the hydrogen conversion device 12 which automatically increases its hydrogen gas output to maintain the level of pressure in the end use application 30. Mode 330 transmits a signal to mode 340 indicating the change in the operational state of valve 34 before transitioning back to mode 310 to repeat the end use application purity monitoring process. Alternatively, mode 330 may utilize other signals to initiate a change in state in valve 34, such signal include but are not limited to electrical current, optical attenuation, infrared light, radio radiation, microwave radiation. The change in state of valve 34 may also be accomplished through computer communications protocols, including but not limited to TCP/IP, RS-232, ModBus and the like. Further, while the change in state described herein is illustrated in discrete steps, it is contemplated that operational state of the valve may also vary continuously from a state where the flow through conduit 32 ceases (e.g. fully closed) to an opposite state where there is no restriction to the hydrogen gas flow (e.g. fully open).

Upon receiving a signal from mode 330 that a change in the operational state of valve 34 has occurred, mode 340 stores the elapsed time tₜ₋₁ that has lapsed since the last change in valve 34 state. Mode 330 then restarts a timer to measure the time of operation for the new valve 34 state. The process then transfers to mode 350 which uses the previous valve state e₁₍ₜ₋₁₎⁻ and time elapsed tₜ₋₁ to calculate the volume hydrogen gas vented with lookup tables 40. From the lookup tables 40, mode 350 stores the amount of hydrogen gas vented Hₙ and transfers to mode 360 where the total hydrogen emissions are calculated. Mode 360 adds the calculated vented volume Hₙ to data previously measured (e.g. Hₙ₋₁) to calculate to total controlled hydrogen emissions Hᵥ from the system 10. The total controlled hydrogen emissions will be determined by summing the values of vented hydrogen gas Hₙ.
Hᵥ=ΣHₙ
or in the case of the embodiment illustrated in FIG. 5:
Hᵥ=ΣHₙ+H_{f}+Hₛ
Depending on the needs of the operator, mode 360 may accumulate the data Hv for periodic reporting (e.g. daily emissions), continuously store the data Hₙ, or upon a request from mode 370. In the case of periodic reporting, mode 360 will pass the data Hv to mode 370 and then transitions back to mode 340 to await a further change in state of valve 34.

Mode 380 monitors and records the current e⁻ being provided by power converter 52 to electrochemical cell stack 54. The current e⁻ is passed to mode 390 which calculates the hydrogen generated using the relationship:
H_{T}=7.52(N)(I)−L, cc/min
Mode 390 accumulates the amount of hydrogen generated HT and periodically passes the data to mode 370 for further processing.

In the exemplary embodiment, mode 370 receives data from modes 390 and mode 360 and uses this data to determine the controlled hydrogen emissions E_{c}:
E_{c}=Hᵥ
Mode 370 also determines the amount of uncontrolled emissions Eᵤ:
Eᵤ=H_{T}—Hᵥ
This data E_{c} and Eᵤ are passed to mode 400 for reporting on either a continuous or period basis to meet the needs of the operator. The reporting may alternately be provided upon request from the operator, a remote computer 48, another mode of process 300 or the like. The request may be made in the form of a signal transmitted to mode 400 or through a manual entry device coupled to a controller 44. Mode 370 then returns to a state waiting for additional data from mode 360 and 390.

The system and method for monitoring, reporting and recording hydrogen gas emissions described herein may also allow operational parameters for the system 10 to be set either remotely or locally. Because the operational parameters can be set remotely, a single operator can monitor and control the amount of controlled and uncontrolled hydrogen gas emissions from virtually any location. The automated and remote monitoring of hydrogen emissions and the setting of operational parameters provides an operational convenience that was previously unattainable with systems of the prior art. In addition, the present invention provides manpower and cost savings over the prior art because a single operator can monitor and operate any number of systems located at different sites. Prior art systems required an operator to be present on-site to monitor and record the emissions data.

The process 300 can be embodied in the form of computer-implemented processes and apparatuses for practicing those processes. The control methods can also be embodied in the form of computer program code containing instructions, embodied in tangible media, such as floppy diskettes, CD-ROMs, hard drives, or any other computer-readable storage medium, wherein, when the computer program code loaded into and executed by a computer, the computer becomes an apparatus for practicing the invention. The process 300 can also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When the implementation is on a general-purpose microprocessor, the computer program code segments configure the microprocessor to create specific logic circuits.

While the invention has been described with reference to a preferred embodiment, it will be understood by those skilled in the art that various changes may be made and equivalents may be substituted for elements thereof without departing from the scope of the invention. For example, the embodiments discussed here are directed to a single end use application, hydrogen conversion device and emission monitoring and reporting system. It is contemplated that the invention described herein may be used in configurations involving multiple end use applications with a single hydrogen conversion device, or multiple hydrogen conversion devices and a single end use application, or any combination thereof. In addition, any modifications may be made to adapt a particular situation or material to the teachings of the invention without departing from the essential scope thereof. Therefore, it is intended that the invention not be limited to the particular embodiment disclosed as the best mode contemplated for carrying out this invention.

What is claimed is:

1. A method for reporting emissions for a process using hydrogen gas, said method comprising:
determining a quantity hydrogen produced by a hydrogen conversion device from a first operating parameter;
determining a quantity of hydrogen purposely released from a second operating parameter;
determining a quantity of uncontrolled hydrogen emissions from said quantity of hydrogen generated and said quantity of hydrogen purposely released;
storing a value representative of said quantity of uncontrolled hydrogen emissions; and,
transmitting said value representative of said quantity of uncontrolled hydrogen emissions.

2. The method for reporting emissions for a process using hydrogen gas of claim 1, said method further comprising the steps of:
determining a quantity of controlled hydrogen emissions from said quantity of hydrogen released;
returning a value indicative of the quantity of controlled hydrogen emissions.

3. The method for reporting emissions for a process using hydrogen gas of claim 2 wherein said value of uncontrolled and controlled hydrogen gas emissions are returned in response to a request.

4. The method for reporting emissions for a process using hydrogen gas of claim 3 wherein said first operating parameter is chosen from a group comprising: amount electrical current provided, amount of water consumed, amount of hydrocarbon material consumed, the amount of coal consumed, amount of natural gas consumed, amount of bio-mass consumed or the amount of methane gas consumed.

5. The method for reporting emissions for a process using hydrogen gas of claim 4 wherein said first operating parameter is the amount of electrical current provided.

6. The method of reporting emissions for a process using hydrogen gas of claim 3 wherein:
said first operating parameter is the amount of current provided to an electrochemical cell stack; and
said quantity of hydrogen produced is determined by multiplying said amount of current by the number of cells in said electrochemical cell stack by a predetermined coefficient.

7. The method of reporting emissions for a process using hydrogen gas of claim 6 wherein said predetermined coefficient is 7.52.

8. The method of reporting emissions for a process using hydrogen gas of claim 6 wherein said predetermined coefficient is 0.037384.

9. A system for reporting emissions from a process using hydrogen gas, said system comprising:
at least one computer processor means for processing data;
storage means for storing data on a storage medium;
a second means for processing data regarding a first hydrogen conversion device operating parameter and data regarding the accumulated amount of hydrogen gas produced;
a third means for processing data regarding a second operating parameter and data regarding the accumulated amount of hydrogen gas purposely released; and,
a fourth means for processing data regarding aggregate controlled and uncontrolled hydrogen emissions that result from the process using hydrogen gas.

10. The system for reporting emissions from a process using hydrogen gas of claim 9, said system further comprising:
a means for measuring a time parameter regarding the second operating parameter;
a means for storing on said storage medium data regarding volumetric flow rates as a function of time.

11. The system for reporting emissions from a process using hydrogen gas of claim 10 wherein said fourth means for processing data aggregates said controlled and uncontrolled hydrogen emissions at least once daily.

12. The system for reporting emissions from a process using hydrogen gas of claim 10, said system further comprising a means for measuring said first operating parameter.

13. The system for reporting emissions from a process using hydrogen gas of claim 12 wherein said first operating parameter is electrical current.

14. The system for reporting emissions from a process using hydrogen gas of claim 12 wherein said first operating parameter is a volume of water feedstock.

15. The system for reporting emissions from a process using hydrogen gas of claim 12 wherein said first operating parameter is a volume of hydrocarbon precursor material.

16. The system for reporting emissions from a process using hydrogen gas of claim 12 further comprising:
a means for retrieving data from a second storage medium; and,
a means for transmitting said aggregate controlled and uncontrolled hydrogen emissions.

17. A system for reporting emissions from a hydrogen cooled electrical generator, said system comprising:
a hydrogen conversion device;
a sensor coupled to said hydrogen conversion device;
a hydrogen cooled electrical generator coupled to said hydrogen conversion device;
a vent coupled to said electrical generator, said vent including a means for limiting hydrogen gas flow through said vent; and,
a first controller electrically coupled to said means for limiting hydrogen gas flow, said first controller including means for determining a quantity of hydrogen gas based on the operating state of said means for limiting hydrogen gas flow.

18. The system for reporting emissions from a hydrogen cooled electrical generator of claim 17 further comprising a second controller coupled to said sensor, said second controller having means for measuring the amount of hydrogen gas produced.

19. The system for reporting emissions from a hydrogen cooled electrical generator of claim 18 further comprising a means for processing data from said first controller and said second controller regarding the aggregate amount of controlled hydrogen gas emissions and uncontrolled hydrogen gas emissions over a predetermined amount of time.

20. The system for reporting emissions from a hydrogen cooled electrical generator of claim 19 wherein said hydrogen conversion device is an electrochemical cell stack.

21. The system for reporting emissions from a hydrogen cooled electrical generator of claim 20 wherein said electrochemical cell stack comprises a plurality of cells each having a polymer membrane and a first and second electrode.

22. The system for reporting emissions from a hydrogen cooled electrical generator of claim 21 wherein said first and second electrodes contain platinum.

23. The system for reporting emissions from a hydrogen cooled electrical generator of claim 20 wherein said sensor measures electrical current supplied to said electrochemical cell stack.

24. The system for reporting emissions from a hydrogen cooled electrical generator of claim 19 wherein said hydrogen conversion device is selected from a group comprised of steam methane reformer, natural gas reformer, coal reformer, hydrocarbon reformer, partial oxidation reactors, ceramic membrane reactor, photolysis reactor, photoelectrolysis reactor, photochemical reactors, photobiological reactors, anaerobic digesters or bio-mass gasification reactors.

25. The system for reporting emissions from a hydrogen cooled electrical generator of claim 19 further comprising a precursor material operably coupled to said hydrogen conversion device, said precursor material being selected from a group comprised of water, algae, bacteria, hydrocarbons, oxygenated hydrocarbons, organic carbon materials, fixed metal hydrides, transferable metal hydrides, and sodium borohydroxide.

26. The system for reporting emissions from a hydrogen cooled electrical generator of claim 24 further comprising a second hydrogen cooled electrical generator coupled to said hydrogen conversion device.

27. The system for reporting emissions from a hydrogen cooled electrical generator of claim 24 further comprising a second hydrogen conversion device coupled to said hydrogen cooled electrical generator.
